(19) United States
(12) Patent Application Publication
(10) Pub. No.: US 2023/0293601 A1
(43) Pub. Date: Sep. 21, 2023

(54) USE OF BACILLUS COAGULANS BC198 OR ITS METABOLITES FOR PREVENTION OR ADJUVANT TREATMENT OF INTESTINAL LESION-RELATED PATHOLOGICAL CHANGES OR FLORA IMBALANCE CAUSED BY CHEMOTHERAPY

(71) Applicant: SYNGEN BIOTECH CO., LTD., Tainan City (TW)

(72) Inventors: Wei-Jen CHEN, Tainan City (TW); Tsung-Yin TANG, Tainan City (TW); Shiuan-Huei WU, Tainan City (TW); Ai-Lun TSENG, Tainan City (TW); Siou-Ru SHEN, Tainan City (TW)

(21) Appl. No.: 17/736,682

(22) Filed: May 4, 2022

(30) Foreign Application Priority Data
Mar. 18, 2022 (TW) 111110119

(51) Int. Cl. A61K 35/742; A61P 31/04; A23L 33/135

(52) U.S. Cl. CPC A61K 35/742; A61P 31/04; A23L 33/135

(57) ABSTRACT

The invention discloses a use of Bacillus coagulans BC198 or its metabolites for prevention or adjuvant treatment of intestinal lesion-related pathological changes or flora imbalance caused by chemotherapy, that is, by administering an effective amount of Bacillus coagulans BC198 to an individual receiving chemotherapy is capable of effectively improving disorders of weight loss, loss of appetite, diarrhea, shortening of length of large intestine, inflammation, intestinal tissues lesion and imbalance of intestinal flora caused by chemotherapeutic drugs, in other words, the Bacillus coagulans BC198 disclosed in the invention can be used as an adjuvant product, a dietetic food product for chemotherapy, or other compositions for improving side effects or enhancing therapeutic effects.

### BACKGROUND OF THE INVENTION

### Field of Invention

The invention relates to a use of probiotics, and more particularly to a use of Bacillus coagulans BC198 or its metabolites for prevention or adjuvant treatment of intestinal lesion-related pathological changes or flora imbalance caused by chemotherapy.

### Related Art

Cancer is the second leading cause of death globally, with an estimated 14 million new cases and 8 million deaths each year, and with an estimated increase of up to 21 million cases diagnosed with cancer by 2030 (BioMed research international, 2018, 2018: 3428437). As far as cancer treatment is concerned, chemotherapy is currently the main clinical treatment method, but about 50-80% of patients receiving chemotherapy will develop intestinal mucositis, and the clinical symptoms are weight loss, decreased appetite, ulcer, diarrhea and abdominal pain, seriously affecting the quality of life of cancer patients (intestinal, 2000, 47: 632-637).

According to research, L-glutamine (L-gln) is an important non-essential amino acid in the gastrointestinal tract, and because it can be used as an essential matrix for epithelial cell nucleic acid synthesis, L-glutamine is often used as an adjuvant drug for patients receiving chemotherapy; however, other studies have shown that L-glutamine is incapable of inhibiting the chemotherapeutic drug doxifluridine-induced diarrhea, or incapable of mitigating severity of the chemotherapeutic drug 5-FU/calciumfolinate-induced stomatitis, nausea and diarrhea (Nutrition, 1997, 13(7-8): 748-751; European journal of cancer, 1999, 35(2): 202-207; Gastroenterology, 2006, 130(2Suppl 1): S106-S116). In conclusion, L-glutamine is capable of reducing the duration of chemotherapy-induced diarrhea in patients, but incapable of improving its severity (Asia Pacific journal of clinical nutrition, 2012, 21(3): 380-385).

Many recent studies have pointed out the importance of intestinal flora for intestinal health, and the administration of chemotherapeutic drugs will affect the imbalance of intestinal flora, which will lead to the occurrence of side effects of chemotherapeutic drugs. Specifically, the chemotherapeutic drug 5-FU will cause the imbalance of intestinal flora accompanied by the occurrence of inflammatory response, which further aggravates intestinal mucositis, which means that the chemotherapeutic drug 5-FU-induced intestinal mucositis is highly correlated with constant of its disordered intestinal flora (Basic & clinical pharmacology & toxicology, 2017, 121(3): 159-168; Frontiers in cellular and infection microbiology, 2017, 7: 455).

It is considered that regulating the intestinal microflora by probiotics is capable of effectively improving the occurrence of side effects caused by chemotherapeutic drugs, but in fact, different strains cause different changes in the composition of intestinal microflora, so the effects of strains on intestinal microflora and clinical symptoms of intestinal mucositis such as body weight, food intake, diarrhea, intestinal tissues lesion and inflammation are unpredictable. For example, studies have shown that administration of Saccharomyces boulardii is incapable of restoring weight loss and decreased food intake induced by the chemotherapeutic drug 5-FU, nor does it capable of mitigating chemotherapeutic drugs-induced intestinal mucosal lesion and increased intestinal permeability (Journal of negative results in biomedicine, 2014, 13: 6).

### SUMMARY OF THE INVENTION

A main object of the invention is to provide a use of Bacillus coagulans BC198 or its metabolites for prevention or adjuvant treatment of intestinal lesion-related pathological changes or flora imbalance caused by chemotherapy, which means that because the Bacillus coagulans BC198 or its metabolites are capable of protecting intestinal cells and reducing an occurrence of lesion induced by chemotherapeutic drugs, maintaining a balance of intestinal flora, maintaining an integrity of intestinal tissues structure, and reducing a production of inflammation-related factors, by administering an effective amount of the Bacillus coagulans BC198 or its metabolites disclosed in the invention to an individual receiving chemotherapy or chemotherapeutic drugs is capable of effectively improving intestinal side effects and related symptoms such as enteritis, diarrhea, weight loss, loss of appetite caused by chemotherapeutic drugs in an individual.

In order to achieve the above object, the invention discloses a use of Bacillus coagulans BC198 or its metabolites for prevention or adjuvant treatment of intestinal lesion-related pathological changes or flora imbalance caused by chemotherapy, which means that by administering an effective amount of the Bacillus coagulans BC198 or its metabolites disclosed in the invention to an individual who is about to receive chemotherapy or has received chemotherapy is capable of effectively improving or mitigating intestinal pathological changes or related side effects caused by chemotherapy.

In one embodiment of the invention, the Bacillus coagulans BC198 are deposited in Taiwan Food Industry Research and Development Institute (FIRDI) with the accession number BCRC910916 and the accession date Jul. 11, 2019; and the German Collection of Microorganisms and Cell Cultures (DSMZ) with the accession date Jul. 10, 2019 and the accession number DSM33206.

In one embodiment of the invention, an effective dose of the Bacillus coagulans BC198 is at least about 5×10⁸ CFU/day administered to each individual daily, any dose within a normal error value range is the so-called effective dose in the invention.

In one embodiment of the invention, the Bacillus coagulans BC198 are prepared as a composition, such as a pharmaceutical composition, a nutritional supplement, a dietary supplement, and the like.

Wherein the composition further contains a glutamine.

In one embodiment of the invention, it is disclosed that the Bacillus coagulans BC198 or its metabolites are a composition for prevention or adjuvant treatment of intestinal lesion-related disorders caused by chemotherapy, wherein the intestinal disorders-related symptoms are weight loss, loss of appetite, diarrhea, enteritis, shortening of length of large intestine, imbalance of intestinal flora, or intestinal tissues lesion such as intestinal mucositis.

In another embodiment of the invention, a use of the Bacillus coagulans BC198 or its metabolites for preparing a composition for regulating a balance of intestinal microflora is disclosed, which means that by administering an effective amount of the Bacillus coagulans BC198 or its metabolites disclosed in the invention to an individual who is about to receive chemotherapy or has received chemotherapy is capable of reducing a number of strains in intestinal tract that are detrimental to intestinal health, and capable of increasing a number of strains in intestinal tract that contribute to intestinal health.

Wherein the strain that is detrimental to intestinal health can cause enteritis, diarrhea, and intestinal tissues pathological changes, and the phylum of the strain that is detrimental to intestinal health is Proteobacteria, Escherichia shigella, Odoribacter, UBA1819 or Staphylococcus.

Wherein the strain that contributes to intestinal health is related to butyric acid production, such as a strain belonging to the phylum Muribaculum or Lachnoclostridium.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the results of analysis of viability of IEC-6 cells treated with different thermo-inactivations of Bacillus followed by cell lesion induced by 5-FU drug.

FIG. 2 shows changes of body weight of mice in each group in embodiment 4 during testing period, wherein the red arrows indicate time points of administration of drug 5-FU.

FIG. 3 shows changes in food intake of the mice in each group in embodiment 4 during testing period, wherein the red arrows indicate time points of administration of drug 5-FU.

FIG. 4 shows the results of analysis of diarrhea index of the mice in each group in embodiment 4, wherein the red arrows indicate time points of administration of drug 5-FU.

FIG. 5 shows the results of assay of concentration of IL-6 in the serum of the mice in each group in embodiment 4.

FIG. 6 shows the results of measuring of length of large intestine of the mice in each group in embodiment 4.

FIG. 7A shows the results of staining the small intestine tissue sections of the mice in each group in embodiment 4 with hematoxylin-eosin.

FIG. 7B shows the results of staining the large intestine tissue sections of the mice in each group in embodiment 4 with hematoxylin-eosin.

FIG. 8 is a histogram of analysis of relative abundance of the top 10 species in fecal samples of the mice in each group in embodiment 4.

FIG. 9 is a clustering heatmap of species abundance obtained by analyzing species abundance in fecal samples of the mice in each group in embodiment 4.

FIG. 10 shows the results of partial least square discriminant analysis (PLS-DA) of fecal samples of the mice in each group in embodiment 4, wherein the percentages in the figure represent contribution rates of the PLS principal components to the sample difference, an average value of the fecal samples in each group is indicated by a center of the normal confidence ellipse, each dot in the figure represents a fecal sample, and the fecal samples in the same group are represented by the same color.

FIG. 11A shows the results of analysis of relative abundance of UBA1819 in the microflora of the mice in each group by metagenomeSeq, wherein the horizontal lines represent the two groups with significant differences, no horizontal line represents that this species has no significant difference between the groups; * represents p value <0.05 between the two groups, ** represents p value <0.01 between the two groups.

FIG. 11B shows the results of analysis of relative abundance of Staphylococcus in the microflora of the mice in each group by metagenomeSeq, wherein the horizontal lines represent the two groups with significant differences, no horizontal line represents that this species has no significant difference between the groups; * represents p value <0.05 between the two groups, ** represents p value <0.01 between the two groups.

FIG. 11C shows the results of analysis of relative abundance of Escherichia shigella in the microflora of the mice in each group by metagenomeSeq, wherein the horizontal lines represent the two groups with significant differences, no horizontal line represents that this species has no significant difference between the groups; * represents p value <0.05 between the two groups, ** represents p value <0.01 between the two groups.

FIG. 11D shows the results of analysis of relative abundance of Muribaculum in the microflora of the mice in each group by metagenomeSeq, wherein the horizontal lines represent the two groups with significant differences, no horizontal line represents that this species has no significant difference between the groups; * represents p value <0.05 between the two groups, ** represents p value <0.01 between the two groups.

### DETAILED DESCRIPTION OF THE INVENTION

The invention discloses a use of Bacillus coagulans BC198 or its metabolites for prevention or adjuvant treatment of intestinal lesion-related pathological changes or flora imbalance caused by chemotherapy; specifically, by administering an effective amount of the Bacillus coagulans BC198 to an individual receiving chemotherapy is capable of effectively improving disorders of weight loss, loss of appetite, diarrhea, shortening of length of large intestine, inflammation, intestinal tissues lesion and imbalance of intestinal flora caused by chemotherapeutic drugs; therefore, the Bacillus coagulans BC198 or its metabolites disclosed in the invention can be prepared as a food, a pharmaceutical composition or a nutritional supplement to prevent or/and treat chemotherapy-induced intestinal mucositis or its related disorders, thereby can be used as an adjuvant product or a dietetic food product for cancer patients.

Further, the Bacillus coagulans BC198 or its metabolites disclosed in the invention are capable of synergizing with glutamine and achieving an effect of greatly improving enteritis, diarrhea, weight loss, loss of appetite or other side effects related to intestinal lesion caused by chemotherapeutic drugs. When the Bacillus coagulans BC198 or its metabolites disclosed in the invention are used together with glutamine, effective doses thereof can be adjusted according to individual species, which are known to a person having ordinary skill in the art to which the invention pertains.

An individual to which the Bacillus coagulans BC198 or its metabolites disclosed in the invention can be administered is an animal of any species, not limited to humans.

“Administration” or “administering” referred to in the invention is not limited to oral administration, and can be eaten with general food or food compositions during administration.

“Composition” referred to in the invention at least contains an effective amount of the Bacillus coagulans BC198 or its metabolites disclosed in the invention, wherein the Bacillus coagulans BC198 comprise heat inactivated bacteria and live bacteria. In addition to the Bacillus coagulans BC198 or its metabolites, the composition can further comprise excipient, vehicle, adjuvant and/or food additive, any pharmaceutically acceptable ingredient or any ingredient acceptable in foods such as protein, saccharide, lipid, carbohydrate, amino acid, vitamin, and/or bacteria with individual safety such as lactic acid bacteria, lactobacillus, yeast, which are well known in the technical field to which the invention pertains. The composition can be a pharmaceutical composition, a food composition, a nutritional supplement, a dietary supplement or an edible article of any form. Dosage forms of the composition include, but are not limited to, spray gas, solution, semi-solid, solid, gelatin capsule, soft capsule, tablet, lozenge, chewing gum and/or freeze-dried powder.

The 16S S rDNA sequence of the Bacillus coagulans BC198 disclosed in the invention is shown in SEQ ID NO: 1, the sequence of the Bacillus coagulans BC198 is compared with the composite sequence comparison database (NCBI blast) for sequence alignment, the results show that the Bacillus coagulans BC198 disclosed in the invention is the closest to Bacillus coagulans strain 4086 with 100.00% similarity. The Bacillus coagulans BC198 disclosed in the invention are deposited in Germany and Taiwan, and the accession information is as follows:

German Collection of Microorganisms and Cell Cultures (DSMZ) with the accession number DSM33206 and the accession date Jul. 10, 2019; and

Taiwan Food Industry Research and Development Institute (FIRDI) with the accession number BCRC910916 and the accession date Jul. 11, 2019.

Culture environment and conditions of the Bacillus coagulans BC198 are: MRS broth medium, pH 6.25, 45° C. anaerobic or aerobic environment.

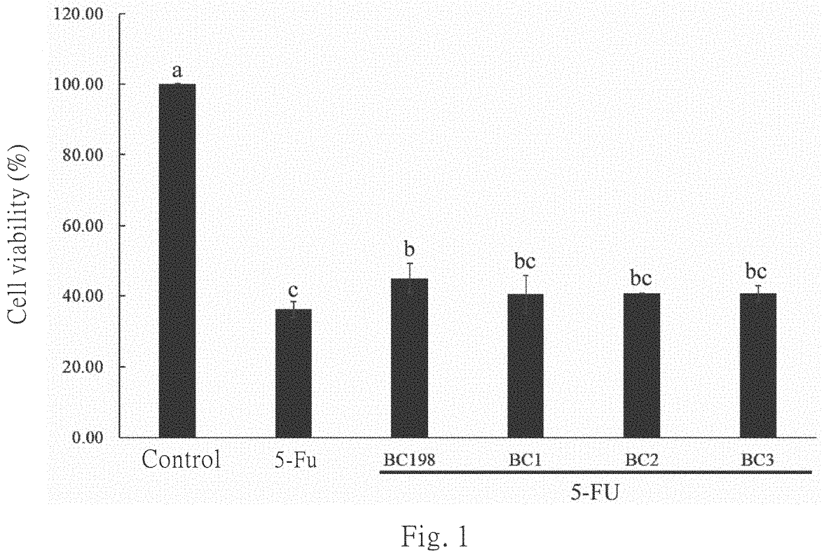

Mycological characteristics of the Bacillus coagulans BC198 include:
1. Cell morphology and Grain staining: when the bacteria are cultured in MRS broth medium under an anaerobic environment at 45° C. for 24 hours, their appearance of a rod-shaped bacillus is observed under a microscope, as shown in FIG. 1.
2. Mobility: with motility.
3. Sporulation: with sporulation.
4. Gram stain: positive.
5. Catalase: negative.

“Effective amount” disclosed in the invention can also be referred to as “effective dose”, which varies according to the biological species to which an administered individual belongs or individual differences. Generally speaking, the effective amount can be determined by experimental results such as dose escalation carried out by a person having ordinary skill in the art to which the invention pertains. For example, the effective amount of daily administration of the Bacillus coagulans BC198 disclosed in the invention to a mammal or a human is at least about 5×10⁸ CFU/day, and an administration period can be 17 days or more. Any amount or period within a normal error range of the aforementioned effective amount or administration period will not affect the Bacillus coagulans BC198 disclosed in the invention in exerting an efficacy on improving clinical disorders of intestinal mucositis caused by chemotherapy in an administered individual body, including weight loss, loss of appetite, diarrhea, and an efficacy on mitigating chemotherapy-induced symptoms including shortening of length of large intestine, inflammatory response, intestinal tissues lesion and imbalance of intestinal flora.

“Chemotherapy” disclosed in the invention, also known as chemo, refers to a treatment method in which a chemotherapeutic drug is administered to an individual suffering from cancer or tumor, wherein the chemotherapeutic drug is a chemically synthesized drug that is capable of entering into an individual body through blood circulation to achieve an efficacy of inhibiting the growth of cancer cells or tumor cells, and further causing the cells to disappear.

In order to explain the technical features and achievable efficacies of the invention, several embodiments will be exemplified in detail hereinafter with reference to the accompanying drawings.

The IEC-6 cell strains with the accession number BCRC60301 of rat normal intestinal epithelial cells (hereinafter referred to as IEC-6 cells) used in the following embodiments are purchased from the Bioresource Collection and Research Center (BCRC) of Taiwan Food Industry Research and Development Institute (FIRDI), and cultured in basal medium DMEM containing 10% serum and insulin at 37° C. and 5% carbon dioxide.

The 5-FU drug (5-Fluouracie) used in the following embodiments is a chemotherapeutic drug.

The experimental data of the following embodiments are expressed as mean±standard deviation (mean±SD). SPSS software is used for statistical analysis, One-way ANOVA is used for assaying, and Duncan's Multiple Range test is used to test the differences between groups of samples. When p-value<0.05, it represents significant difference, and it will be marked with different English letters, such as a, b or c, in the diagram or table.

### Embodiment 1: Isolation of the Bacillus coagulans BC198

Add sterilized water with 10 times a weight of green malt to the green malt, crush it with a homogenizer, settle it by gravity for 10 minutes, put the supernatant into the MRS broth medium, cultivate it at 50° C. for 48 hours, the culture is spread on an MRS agar plate, after anaerobic cultivation at 50° C. for 72 hours, a single colony appeared on the agar medium is collected, and an isolated strain is further obtained by purification, after assaying, its catalase is negative and it is rod-shaped when observed under a microscope.

The DNA of the isolated strain is extracted, the 16S rDNA (ribosomal DNA) fragment is amplified, the obtained PCR product is subjected to agar gel electrophoresis to confirm that the product meets the expected size, and then sequenced to obtain the nucleotide sequence encoded as SEQ ID No: 1, confirming that the isolated strain is indeed the Bacillus coagulans BC198 disclosed in the invention, and through sequence alignment, it can be known that the Bacillus coagulans BC198 is the closest to Bacillus coagulans strain 4086.

### Embodiment 2: Preparation of Viable Bacillus coagulans BC198

The cultured and isolated Bacillus coagulans BC198 are placed in TSB (Tryptone Soy Broth) medium, cultured at 37° C. for 20-24 hours, centrifuged to remove the supernatant, and the bacteria are lyophilized to obtain Bacillus coagulans BC198 live bacteria.

### Embodiment 3: Cell Test

The IEC-6 cells are inoculated into a 96-well plate with 1×10⁴ cells/100 μL culture medium, after the cells are attached on the plate, different thermo-inactivated Bacillus (a number of bacteria is 10⁸ CFU/mL) are added: the Bacillus coagulans BC198 disclosed in the invention, BC1 strains, BC2 strains, and BC3 strains are respectively cultured with 5-FU drug (concentration of 3 μM) for 96 hours, and then MTT solution is added to make a final concentration of each well to be 0.5 mg/mL After culturing for 4 hours in a culture environment of 37° C. and 5% carbon dioxide, aspirate the culture medium, add 100 μL of DMSO (dimethyl sulfoxide), shake in a dark room for 45 minutes, measure the absorbance at 570 nm with a microplate spectrophotometer, and calculate the cell viability of each group. The results are shown in FIG. 1; wherein, the BC1 strains and the BC3 strains are respectively Bacillus coagulans strains isolated from commercially available products, and BC2 are strains from the Taiwan Food Industry Research and Development Institute (FIRDI), with the accession number BCRC 10606.

It can be known from the results in FIG. 1 that, compared with the group without thermo-inactivated bacillus added, after co-cultivation of IEC-6 cells treated with different thermo-inactivated bacillus and 5-FU drug, the cell viability of each group has been improved, showing that although each strain of thermo-inactivated Bacillus has a protective effect on cell lesion induced by the 5-FU drug, comparing the cell viability of each group, it is found that the Bacillus coagulans BC198 disclosed in the invention are capable of significantly improving the cell viability after induction of 5-FU drug. In other words, the Bacillus coagulans BC198 disclosed in the invention have an ability to protect intestinal cells, and are capable of effectively reducing lesions caused by chemotherapeutic drugs to intestinal cells, so as to improve side effects or discomfort caused by chemotherapeutic drugs, such as disorders of loss of body weight, loss of appetite, diarrhea, shortening of length of large intestine, inflammation, intestinal tissues lesion and imbalance of intestinal flora.

### Embodiment 4. Animal Test

A number of 5-week-old male BALB/c strain mice are selected and randomly divided into groups. The experimental period is 18 days. The temperature of rearing environment of the mice in each group is maintained at 22±2° C., with a day and night cycle of 12 hours each. Free feeding for diet is adopted, administration is carried out according to the following conditions, wherein except for the 5-FU drug which is administered on the 11th-13th day of the test, the rest of drugs are administered daily during the test period (that is, from the first day of the test to the end of the test period); 5-FU drug is administered by intraperitoneal injection, and the other drugs are administered orally.
Group 1: control group, with phosphate buffer instead of 5-FU drug;
Group 2: administration of 5-FU drug only (50 mg/kg/day);
Group 3: administration of glutamine (1 g/kg/day) and 5-FU drug (50 mg/kg/day);
Group 4: the Bacillus coagulans BC198 (5×10⁸/day) disclosed in the invention and 5-FU drug (50 mg/kg/day) are administered; and
Group 5: the Bacillus coagulans BC198 (5×108/day) disclosed in the invention, glutamine (1 g/kg/day) and 5-FU drug (50 mg/kg/day) are administered.

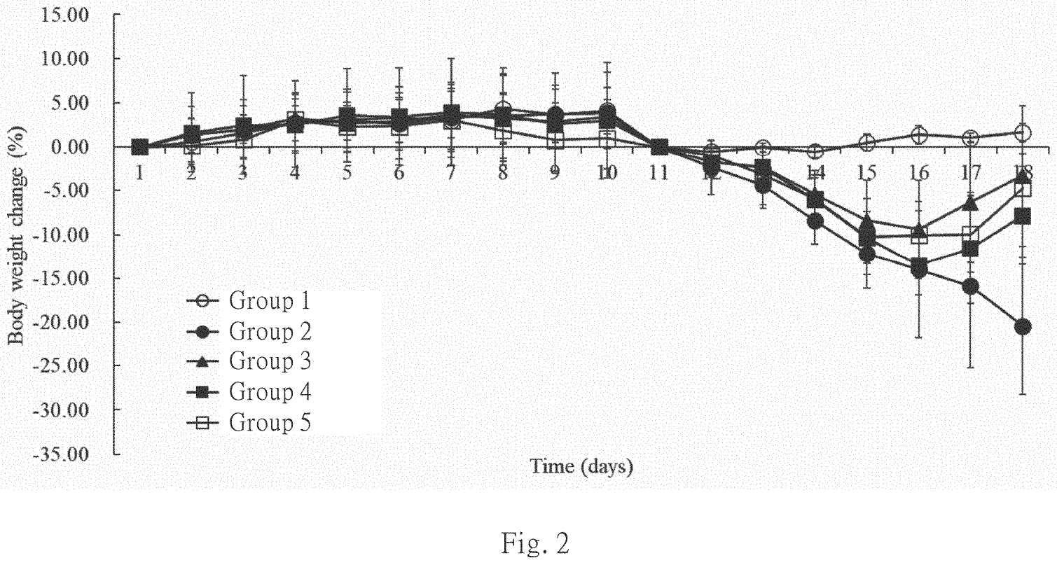

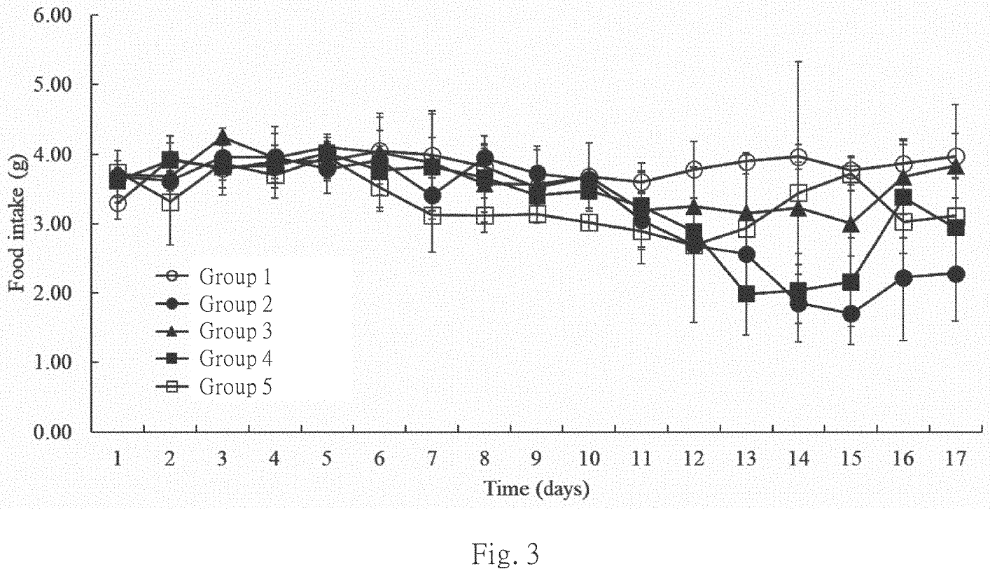

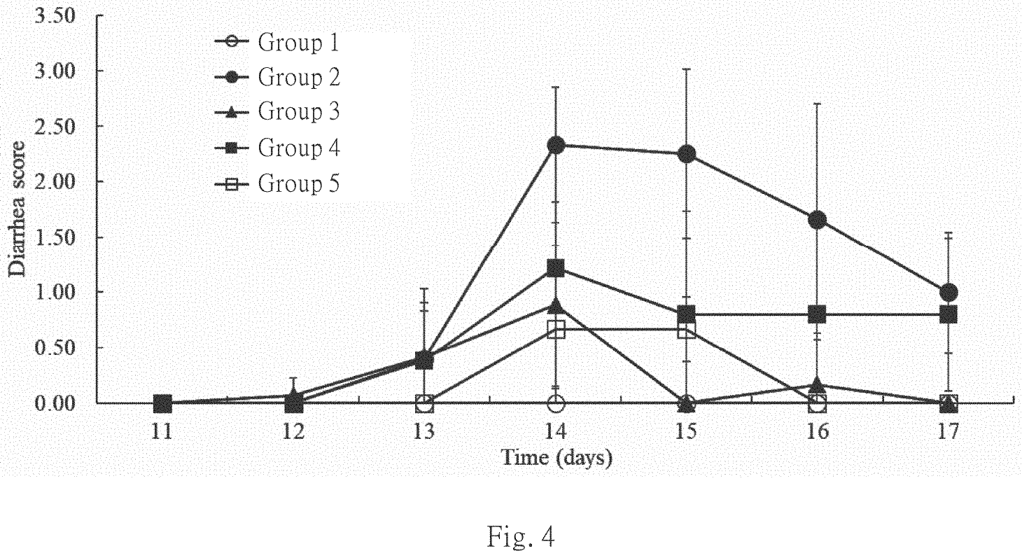

Body weight and feed intake of the mice in each group are measured and recorded every day during the testing period. The results are shown in FIG. 2 and FIG. 3; after administration of 5-FU drug, the feces of the mice in each group are collected, and the diarrhea index of the mice in each group is evaluated, the results are shown in FIG. 4.

It can be known from the results in FIG. 2 and FIG. 3 that after administration of 5-FU on the 11th to 13th day of the test, the body weight and food intake of the mice in group 2 are significantly lower than those in group 1, showing that 5-FU drug indeed causes side effects on an individual, such as decreased appetite and weight loss; the mice in groups 3 and 4 are able to maintain their body weight and food intake respectively; the mice in group 5 can not only maintain their body weight continuously during the test, but also on the 14th-15th days of the test, the food intake is significantly higher than that of the mice in groups 3 and 4.

It can be known from the results in FIG. 4 that after administration of 5-FU drug on the 11-13 days of the test, the diarrhea index of the mice in group 2 on the 14-15th days of the test is significantly increased, indicating that the 5-FU drug can cause lesion to the intestinal cells, causing an individual to experience disorders such as diarrhea or gastrointestinal discomfort; compared with the mice in group 2, the mice in groups 3 and 4 are able to alleviate the diarrhea caused by 5-FU drug; and the mice in group 5 can effectively reduce the diarrhea index from the 14th day of the test.

The results from FIG. 2 to FIG. 4 show that administration of the Bacillus coagulans BC198 disclosed in the invention before an individual treated with chemotherapy is capable of effectively improving disorders of weight loss, loss of appetite, diarrhea, gastrointestinal discomfort caused by chemotherapeutic drugs or chemotherapy, and when the Bacillus coagulans BC198 disclosed in the invention are used in combination with glutamine, side effects caused by gastrointestinal discomfort and intestinal cell lesion caused by chemotherapeutic drugs can be greatly improved or alleviated.

### Embodiment 5: Analysis of Effects of the Bacillus coagulans BC198 on Inflammation-Related Cytokines

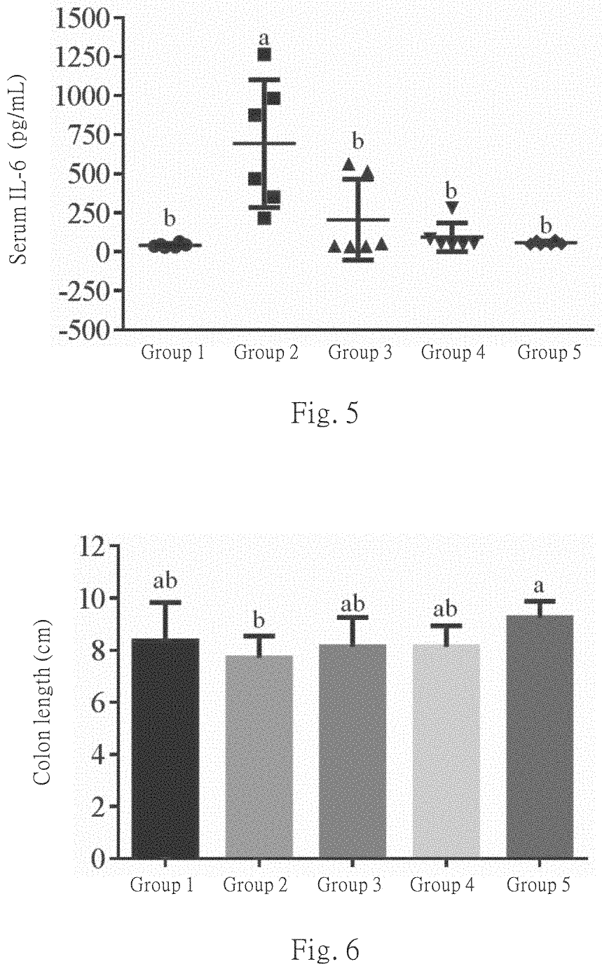

After the test period in embodiment 4, the serum of the mice in each group is collected respectively, and the concentration of IL-6 in the serum of the mice in each group is analyzed with a commercial kit. The results are shown in FIG. 5.

From the results in FIG. 5, it can be known that the concentration of IL-6 in the serum of the mice in group 2 is significantly higher than that of the mice in group 1, indicating that the 5-FU drug can indeed lead to an occurrence of inflammatory reactions; the concentrations of IL-6 in the serum of the mice in groups 3 to 5 are significantly lower than that of the mice in group 2 respectively, and the concentration of IL-6 in the serum of the mice in group 5 is lower than that of the mice in group 3.

The results of FIG. 5 show that the Bacillus coagulans BC198 disclosed in the invention are reliably capable of reducing an occurrence of inflammatory reactions caused by chemotherapy or chemotherapeutic drugs, and the Bacillus coagulans BC198 disclosed in the invention are capable of synergizing with glutamine to be capable of strengthening an ability of inhibiting production of inflammation-related cytokines to achieve an efficacy of effectively alleviating or improving inflammation caused by chemotherapeutic drugs or its related side effects.

### Embodiment 6: Analysis of Effects of the Bacillus coagulans BC198 on Intestinal Tissues

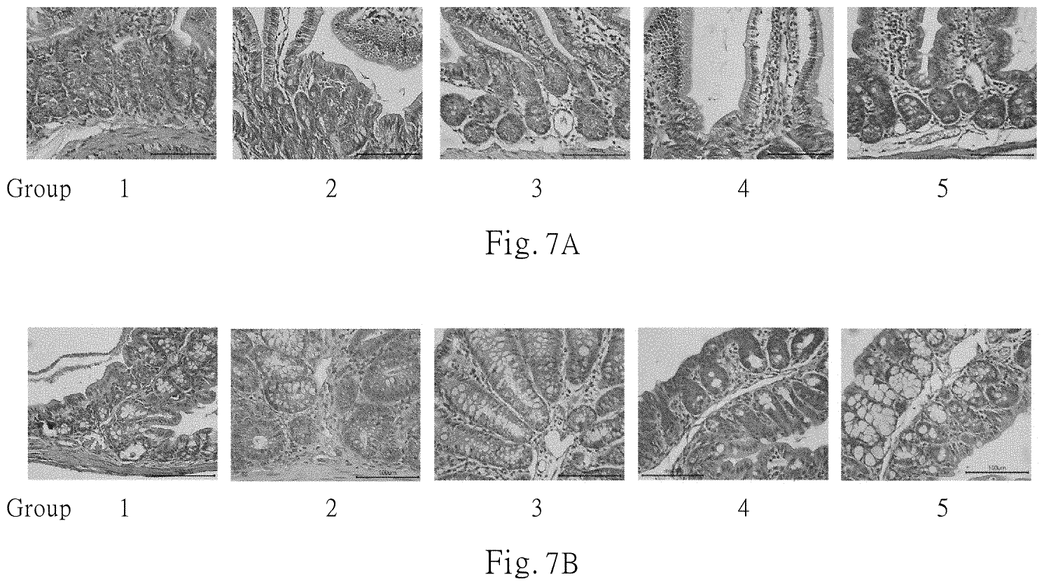

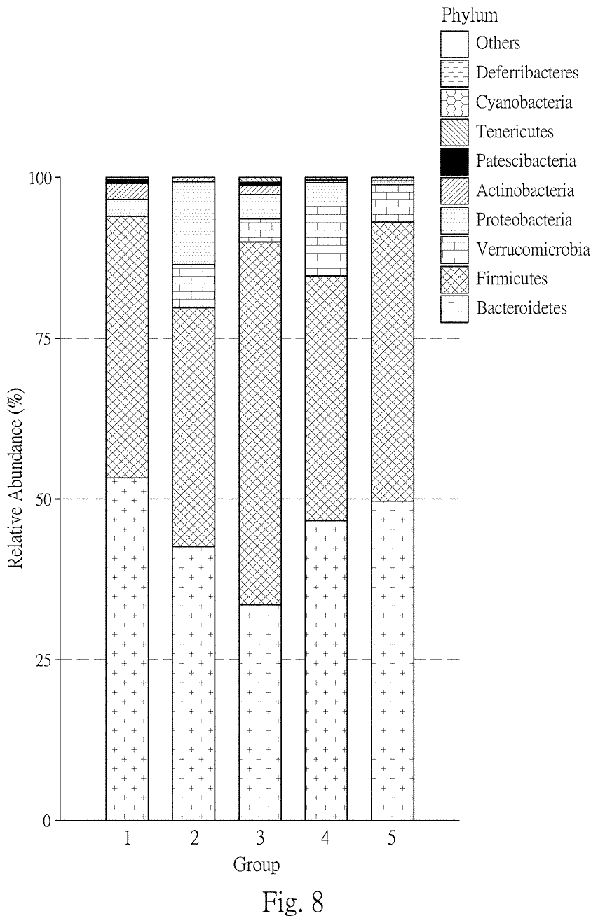

After the test period in embodiment 4, large intestine and small intestine tissues of the mice in each group are collected, and a length of the large intestine of the mice in each group is measured respectively, the results are shown in FIG. 6; the large intestine and small intestine of the mice in each group are sliced respectively, and stained with hematoxylin-eosin, the results are shown in FIGS. 7A and 7B.

From the results in FIG. 6, it can be known that compared with the mice in group 1, the length of the large intestine of the mice in group 2 has a tendency to shorten, the mice in group 3 and group 4 maintain their large intestine lengths, and the mice in group 5 not only can maintain the lengths of their large intestines, but are able to increase the lengths of their large intestines. Further, it can be known from the results in FIG. 5 and FIG. 6 that by continuously administrating the Bacillus coagulans BC198 disclosed in the invention in advance to an individual receiving chemotherapeutic drugs is capable of effectively alleviating or improving side effects of diarrhea or intestinal discomfort caused by chemotherapeutic drugs in an early stage by maintaining a length of the large intestine.

From the results in FIGS. 7A and 7B, it can be known that, compared with the mice in group 1, the structure of the small intestine tissues of the mice in group 2 is lesioned, such as shortened villi, and the large intestinal tissues have obvious pathological changes, such as shortened villi and disappeared glandular fossa (indicated by the red arrows); the mice in groups 3 and 4 are able to mitigate lesion to the structures of the small and large intestines induced by 5-FU drug respectively, which means that the structures of the small and large intestines of the mice in groups 3 and 4 are relatively intact compared with those of the mice in group 2; while the small intestine tissues and large intestine tissues of the mice in group 5 are not only protected from being lesioned by the 5-FU drug to avoid an occurrence of pathological changes in the small intestine or large intestine, but also the structures of the small intestine tissues and the large intestine tissues can be maintained, such as maintaining an integrity of the small intestinal villus tissues, and maintaining an integrity and a number of the glandular fossae.

The results from FIGS. 6 to 7 show that by continuously administrating the Bacillus coagulans BC198 disclosed in the invention alone in advance to an individual receiving chemotherapy or chemotherapeutic drugs is capable of reducing lesion on the intestinal tissues caused by chemotherapeutic drugs during chemotherapy of an individual to achieve an efficacy of maintaining an integrity of the intestinal structure or reducing gastrointestinal disorders caused by chemotherapeutic drugs; and, the Bacillus coagulans BC198 disclosed in the invention are capable of producing synergistic effects with glutamine, which means that when the Bacillus coagulans BC198 disclosed in the invention and glutamine are simultaneously administered to an individual receiving chemotherapy or chemotherapeutic drugs, a protective effect on the small intestine and the large intestine can be strengthened to achieve efficacies of protecting the intestinal tissues, maintaining the structural integrity of the intestinal tract, and preventing pathological changes in the intestinal tissues effectively.

### Embodiment 7: Microflora Analysis

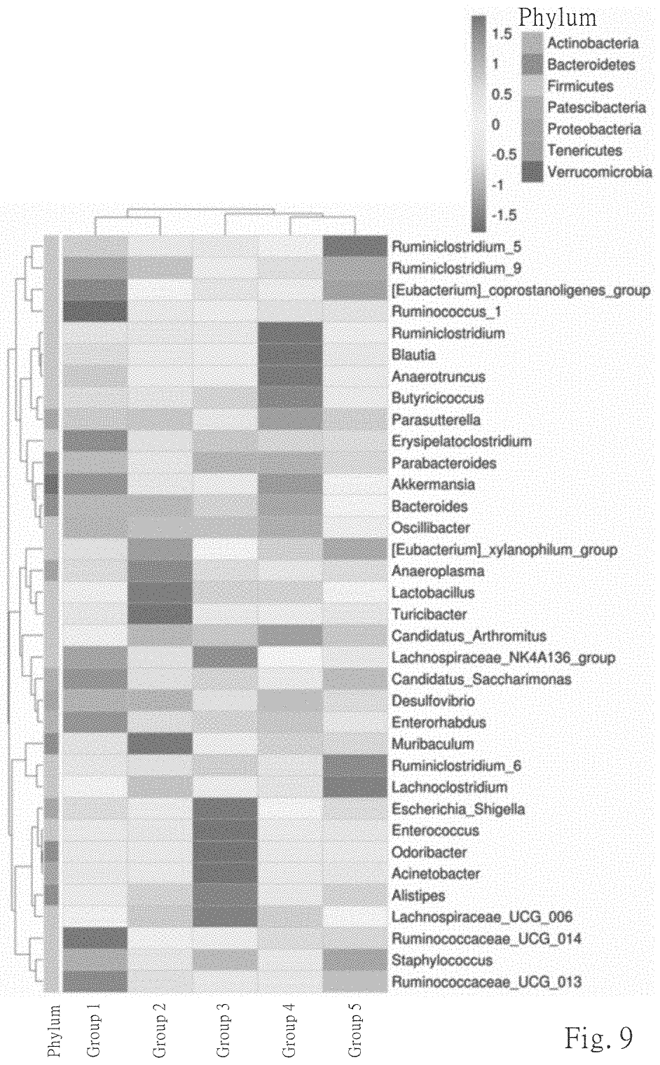

After the test in embodiment 4, the feces of the mice in each group are collected, and sequence analysis of the feces of the mice in each group is outsourced, and then similarity (greater than 97%) OTUs (Operational Taxonomic Units) clustering and species classification analysis are performed. According to the species annotation results, the top 10 species with relative abundance in the mice in each group in the phylum are selected. The results are shown in FIG. 8. Wherein fecal sequencing analysis and microflora analysis are known to a person having ordinary skill in the art to which the invention pertains; generally speaking, the fecal samples are firstly performed with DNA extraction and purification, followed by PCR amplification, purification and sequencing, the sequencing results will go through steps of paired sequence splicing (Raw Tags) and filtering to obtain identifiable valid data, and then OTUs clustering and species classification analysis are performed.

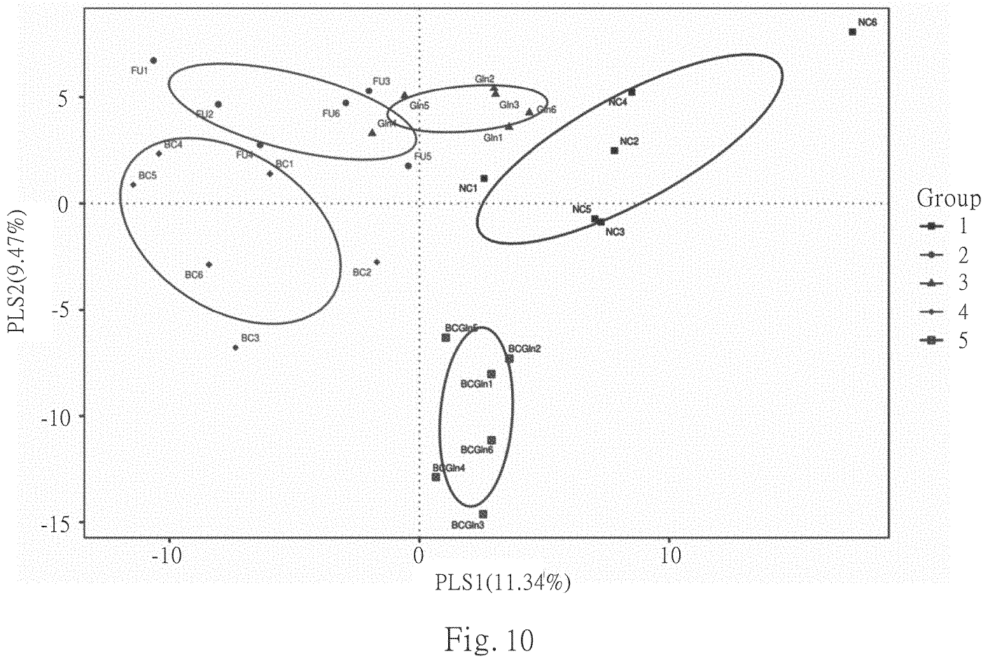

According to the species annotation information and abundance information of all the groups in each taxonomic rank, the top 35 genera in abundance are selected, and the group abundance is the average abundance of all the samples in the group. Clustering is performed at the species level to draw a species heatmap of the fecal samples in each group, the results are shown in FIG. 9.

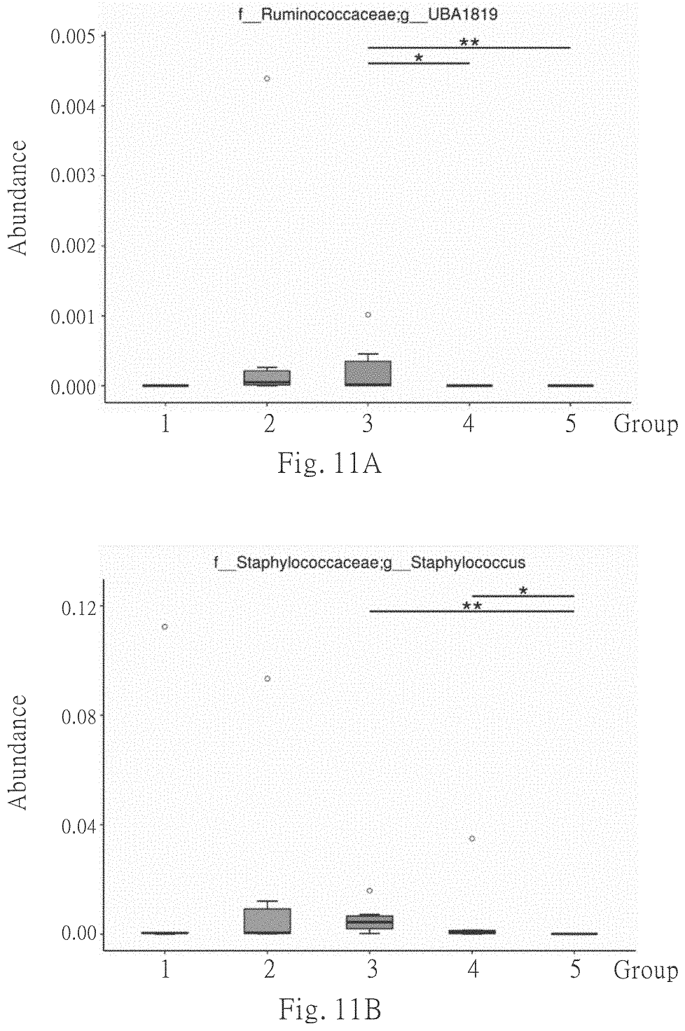

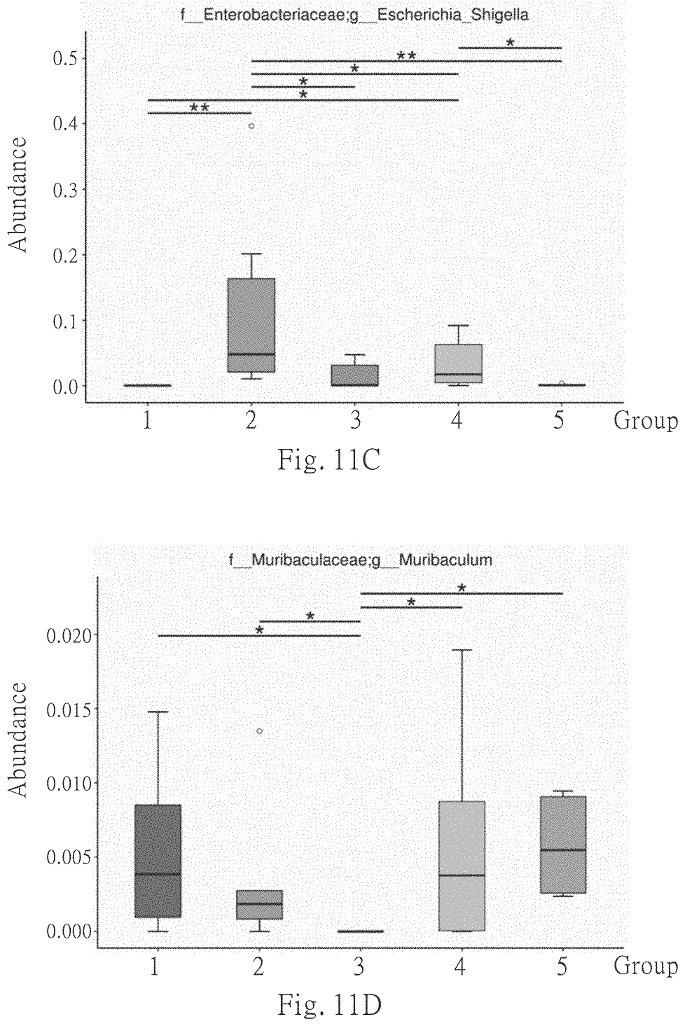

Partial least square discriminant analysis (PLS-DA) is used to analyze the β-diversity of microbial flora in the feces of the mice in each group to determine whether there is a difference in bacterial flora among the microflora of the mice in each group. The results are shown in FIG. 10. The microflora data of the mice in each group are analyzed by metagenomeSeq to evaluate specific differences between the microflora of the mice in each group, and the results are shown in FIG. 11A to FIG. 11D.

As shown in FIG. 8, when the intestinal microflora of group 1 without characterization of intestinal mucosa is regarded as a normal microflora, in the feces of the mice in group 2, the relative abundance of the mucosa-associated inflammation-driving microflora: Proteobacteria is relatively higher, because previous studies have pointed out that increase in the abundance of Proteobacteria will lead to an imbalance of intestinal microflora, which means that increase in the abundance of Proteobacteria can be used as a microbial characteristic for determining intestinal and epithelial cell dysfunction. Therefore, the intestinal microflora of the mice in group 2 is in an unbalanced state, and it is inferred that the mice in group 2 have intestinal and epithelial cell dysfunction; compared with the mice in group 2, the relative abundance of Proteobacteria in the mice in groups 3 to 5 can be reduced, wherein a degree of reduction in the relative abundance of Proteobacteria in the mice in group 5 is the highest.

From the results in FIG. 9, it can be known that the microflora distribution of the mice in group 2 is significantly different from that of the mice in group 1, which confirms that the intestinal microflora of the mice in group 2 already shows an unbalanced state, and the abundance of the microflora associated with diarrhea and enteritis in the mice in group 2 is significantly increased, such as Escherichia shigella and Odoribacter; while the abundances of the microflora associated with diarrhea and enteritis in the mice in groups 3 to 5 are lower than that of the mice in group 2, and, the relative abundance of the butyric acid-producing strain: Lachnoclostridium increases in the microflora of the mice in group 5.

It can be known from the results in FIG. 10 that the microflora distribution of the mice in group 2 is far away from the microflora of the mice in group 1, indicating that the microflora of the mice in group 2 already shows an unbalanced state; the microflora distributions of the mice in groups 3 and 4 are close to that of the mice in group 2, wherein a small part of the microflora distribution of the mice in group 3 overlaps with group 2, indicating that there are similar microflora in the intestines of the mice in group 3 and group 2; however, the microflora distribution of the mice in group 5 is significantly different from that of the mice in group 2, indicating that by simultaneously administering the Bacillus coagulans BC198 disclosed in the invention and glutamine is capable of effectively improving imbalance of intestinal flora caused by chemotherapeutic drugs.

As shown in FIG. 11A to FIG. 11B, compared with group 1, the relative abundances of UBA1819 and Staphylococcus in the microflora of group 2 significantly increase; compared with group 2, the relative abundances of UBA1819 and Staphylococcus in group 3 and group 4 can not be significantly reduced, but the relative abundances of UBA1819 and Staphylococcus in group 5 can be significantly reduced. As shown in FIG. 11C, compared with group 1, the relative abundance of Escherichia shigella is significantly increased in the microflora of group 2; compared with group 2, the relative abundances of Escherichia shigella in group 3 to group 5 can be significantly reduced respectively, wherein a degree of reduction in group 5 is the highest. Also, as shown in FIG. 11D, compared with group 1, the relative abundance of Muribaculum in the microflora of group 2 is significantly reduced; compared with group 2, the relative abundance of Muribaculum in group 3 is significantly reduced; compared with group 3, the relative abundances of Muribaculum in groups 4 and 5 are significantly increased respectively, wherein an extent of increase in group 5 is more significant.

As known by a person having ordinary skill in the art to which the invention pertains, Escherichia shigella is a strain associated with diarrhea; Staphylococcus is a β-glucuronidase-producing strain, which can induce intestinal mucositis; UBA1819 is a strain associated with driving an occurrence of enteritis; and the results from FIG. 8 to FIG. 11 show that by continuously administrating the Bacillus coagulans BC198 disclosed in the invention in advance to an individual receiving chemotherapy is capable of maintaining a balance of intestinal microflora of the individual, capable of effectively reducing a abundance of strains associated with promoting enteritis or diarrhea, such as Escherichia shigella, Staphylococcus, UBA1819, and capable of increasing a relative abundance of the butyric acid-producing strain: Muribaculum; and when the Bacillus coagulans BC198 disclosed in the invention is used together with glutamine, effects of reducing a relative abundance of the above-mentioned strains that are detrimental to intestinal health and increasing a relative abundance of the strains that are beneficial to intestinal health are more significant. It can be known that the Bacillus coagulans BC198 disclosed in the invention are capable of reliably maintaining a balance of intestinal microflora, capable of reducing or inhibiting the growth of strains in an intestinal tract related to enteritis or diarrhea, and capable of promoting the growth of strains that are beneficial to an environment of digestive system, thereby effectively improving side effects such as enteritis, weight loss, diarrhea, loss of appetite caused by an individual receiving chemotherapy; in addition, the Bacillus coagulans BC198 disclosed in the invention are capable of producing synergistic effects with glutamine, which means that by administrating the Bacillus coagulans BC198 disclosed in the invention and glutamine simultaneously is capable of producing a better effect on reducing an imbalance of intestinal flora caused by the chemotherapeutic drug 5-FU.

What is claimed is:

1. A method for prevention or adjuvant treatment of an intestinal lesion-related disorder caused by chemotherapy, comprising administering a composition containing an effective amount of Bacillus coagulans BC198 to an individual, and administering the Bacillus coagulans BC198 at a time point before or at the same time the individual receiving a chemotherapeutic drug being capable of effectively preventing or improving a disorder related to intestinal lesion caused by the chemotherapeutic drug; wherein the Bacillus coagulans BC198 are deposited in the German Collection of Microorganisms and Cell Cultures (DSMZ) with the accession date Jul. 10, 2019 and the accession number DSM33206.

2. The method for prevention or adjuvant treatment of the intestinal lesion-related disorder caused by chemotherapy as claimed in claim 1, wherein the intestinal lesion-related disorder is an intestinal mucositis.

3. The method for prevention or adjuvant treatment of the intestinal lesion-related disorder caused by chemotherapy as claimed in claim 1, wherein the intestinal lesion-related disorder has symptoms of weight loss, loss of appetite, diarrhea, enteritis, shortening of length of large intestine, imbalance of intestinal flora, or intestinal tissues lesion.

4. The method for prevention or adjuvant treatment of the intestinal lesion-related disorder caused by chemotherapy as claimed in claim 1, wherein the composition further comprises a glutamine.

5. The method for prevention or adjuvant treatment of the intestinal lesion-related disorder caused by chemotherapy as claimed in claim 1, wherein an effective amount of the Bacillus coagulans BC198 is at least 5×10⁸ CFU/day administered to each individual daily.

6. The method for prevention or adjuvant treatment of the intestinal lesion-related disorder caused by chemotherapy as claimed in claim 2, wherein an effective amount of the Bacillus coagulans BC198 is at least 5×10⁸ CFU/day administered to each individual daily.

7. The method for prevention or adjuvant treatment of the intestinal lesion-related disorder caused by chemotherapy as claimed in claim 3 wherein an effective amount of the Bacillus coagulans BC198 is at least 5×10⁸ CFU/day administered to each individual daily.

8. The method for prevention or adjuvant treatment of the intestinal lesion-related disorder caused by chemotherapy as claimed in claim 4, wherein an effective amount of the Bacillus coagulans BC198 is at least 5×10⁸ CFU/day administered to each individual daily.

9. A method for regulating balance of intestinal microflora, comprising administering a composition containing an effective amount of Bacillus coagulans BC198 to an individual, and administering the Bacillus coagulans BC198 at a time point before or at the same time the individual receiving a chemotherapeutic drug being capable of effectively preventing or improving an imbalance of intestinal microflora or its related disorders caused by the chemotherapeutic drug; wherein the Bacillus coagulans BC198 are deposited in the German Collection of Microorganisms and Cell Cultures (DSMZ) with the accession date Jul. 10, 2019 and the accession number DSM33206.

10. The method for regulating balance of intestinal microflora as claimed in claim 9, wherein the composition is used for inhibiting a growth of at least one strain in an intestinal tract which is associated with an occurrence of enteritis or/and diarrhea, and the phylum of the strain associated with an occurrence of enteritis or/and diarrhea is Proteobacteria, Escherichia shigella, Odoribacter, UBA1819 or Staphylococcus.

11. The method for regulating balance of intestinal microflora as claimed in claim 9, wherein the composition is used for promoting a growth of at least one strain related to butyric acid production in an intestinal tract, and the phylum of the strain related to butyric acid production is Muribaculum or Lachnoclostridium.

12. The method for regulating balance of intestinal microflora as claimed in claim 9, wherein the composition further comprises a glutamine.

13. The method for regulating balance of intestinal microflora as claimed in claim 9, wherein an effective dose of the Bacillus coagulans BC198 is at least 5×10⁸ CFU/day administered to each individual daily.

14. The method for regulating balance of intestinal microflora as claimed in claim 10, wherein an effective dose of the Bacillus coagulans BC198 is at least 5×10⁸ CFU/day administered to each individual daily.

15. The method for regulating balance of intestinal microflora as claimed in claim 11, wherein an effective dose of the Bacillus coagulans BC198 is at least 5×10⁸ CFU/day administered to each individual daily.

16. The method for regulating balance of intestinal microflora as claimed in claim 12, wherein an effective dose of the Bacillus coagulans BC198 is at least 5×10⁸ CFU/day administered to each individual daily.


SEQUENCE LISTING (1 sequence)

SEQ ID NO: 1 - DNA, 891 bp, Bacillus coagulans
tgcagtcgtgcggaccttttaaaagcttgcttttaaaaggttagcggcggacgggtgagtaacacgtgggcaacctgcctgtaagatcgggataacgccgggaaaccggggctaataccggatagttttttcctccgcatggaggaaaaaggaaagacggcttttgctgtcacttacagatgggcccgcggcgcattagctagttggtggggtaacggctcaccaaggcaacgatgcgtagccgacctgagagggtgatcggccacattgggactgagacacggcccaaactcctacgggaggcagcagtagggaatcttccgcaatggacgaaagtctgacggagcaacgccgcgtgagtgaagaaggccttcgggtcgtaaaactctgttgccggggaagaacaagtgccgttcgaacagggcggcgccttgacggtacccggccagaaagccacggctaactacgtgccagcagccgcggtaatacgtaggtggcaagcgttgtccggaattattgggcgtaaagcgcgcgcaggcggcttcttaagtctgatgtgaaatcttgcggctcaaccgcaagcggtcattggaaactgggaggcttgagtgcagaagaggagagtggaattccacgtgtagcggtgaaatgcgtagagatgtggaggaacaccagtggcgaaggcggctctctggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggccgcaaggctgaactcaaaggaattgacggggcccgcacaag